SEQUENCE LISTING (21 sequences)

SEQ ID NO: 1 - protein, 20 aa, Artificial Sequence
  Description of Artificial Sequence Synthetic peptide
MDAMKRGLCCVLLLCGAVFV

SEQ ID NO: 2 - DNA, 60 bp, Artificial Sequence
  Description of Artificial Sequence Synthetic oligonucleotide
atggatgcaatgaagaggggcctgtgctgcgtgctgctgctgtgtggcgccgtgtttgtg

SEQ ID NO: 3 - DNA, 60 bp, Artificial Sequence
  Description of Artificial Sequence Synthetic oligonucleotide
atggatgccatgaagagaggactgtgctgcgtgctgctgctctgtggagccgtctttgtg

SEQ ID NO: 4 - protein, 20 aa, Artificial Sequence
  Description of Artificial Sequence Synthetic peptide
SPSEITRAPRCPAVRSLLRS

SEQ ID NO: 5 - DNA, 60 bp, Artificial Sequence
  Description of Artificial Sequence Synthetic oligonucleotide
agccctagcgagatcaccagagcccccagatgccctgccgtgagaagcctgctgcggagc

SEQ ID NO: 6 - DNA, 60 bp, Artificial Sequence
  Description of Artificial Sequence Synthetic oligonucleotide
agccctagcgagatcacccgggctcccagatgccctgccgtccggagcctgctgcggagc

SEQ ID NO: 7 - DNA, 35 bp, Artificial Sequence
  Description of Artificial Sequence Synthetic oligonucleotide
ctagtattcccctgacccgccataacaggcccggc

SEQ ID NO: 8 - DNA, 28 bp, Artificial Sequence
  Description of Artificial Sequence Synthetic oligonucleotide
ctcatggttacccaggcgaagccaggta

SEQ ID NO: 9 - DNA, 3798 bp, Artificial Sequence
  Description of Artificial Sequence Synthetic polynucleotide
agatctgatatcgccaccatggatgcaatgaagaggggcctgtgctgcgtgctgctgctgtgtggcgccgtgtttgtgagccctagcgagatcaccagagcccccagatgccctgccgtgagaagcctgctgcggagccggtacagagaagtgtggcccctggccacctttgtgaggagactgggccctgagggcaggagactggtgcagcctggcgaccccaaaatctacaggaccctggtggcccagtgtctggtgtgtatgcactggggcagccagccccctcccgccgacctgagcttccaccaggtgtccagcctgaaggaactggtggccagagtggtgcagagactgtgcgagcggaacgagagaaacgtgctggccttcggcttcgagctgctgaacgaggccagaggcggccctcccatggccttcaccagctctgtgaggagctacctgcccaacaccgtgatcgagaccctgagagtgagcggcgcctggatgctgctgctgagcagagtgggcgatgacctgctggtgtacctgctggcccactgcgccctgtatctgctggtgccccccagctgcgcctaccaggtgtgcggatcccccctgtaccagatttgcgccaccaccgacatctggcccagcgtgtctgccagctacagacccaccagacctgtgggccggaacttcaccaacctgcggttcctgcagcagatcaagagcagcagcagacaggaggcccccaagcccctggccctgcccagcagaggcaccaagagacacctgagcctgaccagcaccagcgtgcccagcgccaagaaagccagatgctaccccgtgcctagagtggaggagggccctcacagacaggtgctgcccacccccagcggcaagagctgggtgcccagccccgccagaagccccgaagtgcccaccgccgagaaggacctgagcagcaagggcaaagtgagcgacctgtctctgagcggcagcgtgtgttgcaagcacaagcccagcagcaccagcctgctgagcccccccagacagaacgccttccagctgaggcctttcatcgagacccggcacttcctgtacagcagaggcgatggccaggagagactgaaccccagcttcctgctgagcaacctgcagcctaacctgaccggcgccagacgcctggtggagatcatcttcctgggcagcagacccagaaccagcggccctctgtgcagaacccaccggctgagcaggcggtactggcagatgagacccctgttccagcagctgctggtgaaccacgccgagtgccagtatgtgcggctgctgaggagccactgcagattcaggaccgccaaccagcaggtgaccgacgccctgaacaccagcccccctcacctgatggatctgctgaggctgcacagcagcccctggcaggtgtacggcttcctgagagcctgcctgtgcaaagtggtgtccgccagcctgtggggcaccagacacaacgagcggcggttcttcaagaatctgaagaagttcatcagcctgggcaagtacggcaagctgagcctgcaggaactgatgtggaagatgaaagtggaggactgccactggctgagaagcagccccggcaaggacagagtgcctgccgccgagcacagactgagggagagaatcctggccacattcctgttctggctgatggacacctacgtggtgcagctgctgcggtccttcttctacatcaccgagagcaccttccagaagaaccggctgttcttctaccggaagtctgtgtggagcaagctgcagagcatcggagtgagacagcacctggagagagtgaggctgagagagctgagccaggaggaagtgagacaccaccaggatacctggctggccatgcccatctgccggctgagattcatccccaagcccaacggcctgagacccatcgtgaacatgagctacagcatgggcacaagagccctgggcagaagaaagcaggcccagcacttcacccagcggctgaaaaccctgttctccatgctgaactacgagcggaccaagcacccacacctgatgggcagcagcgtgctgggcatgaacgacatctaccggacctggagagccttcgtgctgagagtgcgggccctggaccagacccctcggatgtacttcgtgaaggccgccatcaccggcgcctacgacgccatcccccagggcaaactggtggaagtggtggccaacatgatcaggcacagcgagtccacctactgcatcaggcagtacgccgtggtgagaagagacagccagggccaggtgcacaagagcttccggagacaggtgaccaccctgagcgatctgcagccttacatgggccagttcctgaagcacctgcaggatagcgacgccagcgccctgagaaatagcgtggtgatcgagcagagcatcagcatgaacgagtccagcagcagcctgttcgacttcttcctgcacttcctgaggcacagcgtggtgaagatcggcgacagatgctacacccagtgtcagggcatccctcagggctctagcctgagcaccctgctgtgtagcctgtgcttcggcgacatggagaataagctgttcgccgaagtgcagagagatggcctgctgctgcgcttcgtggacgatttcctgctggtgaccccacacctggaccaggccaagaccttcctgagcacactggtgcacggcgtgcccgagtacggctgcatgatcaatctgcagaaaaccgtggtgaacttccctgtggagcccggcaccctgggcggagccgccccttaccagctgcccgcccactgcctgttcccctggtgcggactgctgctggatacccagaccctggaagtgttctgcgactacagcggctacgcccagaccagcatcaagaccagcctgaccttccagagcgtgttcaaggccggcaagaccatgaggaacaagctgctgagcgtgctgagactgaagtgccacggcctgttcctggatctgcaggtgaacagcctgcagaccgtgtgtatcaacatctacaagattttcctgctgcaggcctacagattccacgcctgcgtgatccagctgcccttcgaccagagagtgcggaagaacctgaccttcttcctggggatcatcagcagccaggccagctgctgctacgccatcctgaaagtgaagaaccccggcatgaccctgaaggccagcggcagcttccctcccgaggccgcccactggctgtgctaccaggcctttctgctgaagctggccgcccacagcgtgatctacaagtgcctgctgggccctctgagaaccgcccagaagctgctgtgccggaagctgcccgaggccaccatgaccattctgaaagccgccgccgaccccgccctgagcaccgacttccagaccatcctggactctagagcccctcagagcatcaccgagctgtgcagcgagtaccggaacacccagatttacaccatcaacgacaagatcctgagctacaccgagtctatggccggcaagcgggagatggtgatcatcaccttcaagagcggcgccacctttcaggtggaagtgcctggcagccagcacatcgacagccagaagaaggccatcgagcggatgaaggacaccctgcggatcacctacctgaccgagaccaagatcgacaagctgtgtgtgtggaacaacaagacccccaacagcatcgccgccatctctatggagaactgatctagaaattaagtcgacgaattc

SEQ ID NO: 10 - protein, 1251 aa, Artificial Sequence
  Description of Artificial Sequence Synthetic polypeptide
MDAMKRGLCCVLLLCGAVFVSPSEITRAPRCPAVRSLLRSRYREVWPLATFVRRLGPEGRRLVQPGDPKIYRTLVAQCLVCMHWGSQPPPADLSFHQVSSLKELVARVVQRLCERNERNVLAFGFELLNEARGGPPMAFTSSVRSYLPNTVIETLRVSGAWMLLLSRVGDDLLVYLLAHCALYLLVPPSCAYQVCGSPLYQICATTDIWPSVSASYRPTRPVGRNFTNLRFLQQIKSSSRQEAPKPLALPSRGTKRHLSLTSTSVPSAKKARCYPVPRVEEGPHRQVLPTPSGKSWVPSPARSPEVPTAEKDLSSKGKVSDLSLSGSVCCKHKPSSTSLLSPPRQNAFQLRPFIETRHFLYSRGDGQERLNPSFLLSNLQPNLTGARRLVEIIFLGSRPRTSGPLCRTHRLSRRYWQMRPLFQQLLVNHAECQYVRLLRSHCRFRTANQQVTDALNTSPPHLMDLLRLHSSPWQVYGFLRACLCKVVSASLWGTRHNERRFFKNLKKFISLGKYGKLSLQELMWKMKVEDCHWLRSSPGKDRVPAAEHRLRERILATFLFWLMDTYVVQLLRSFFYITESTFQKNRLFFYRKSVWSKLQSIGVRQHLERVRLRELSQEEVRHHQDTWLAMPICRLRFIPKPNGLRPIVNMSYSMGTRALGRRKQAQHFTQRLKTLFSMLNYERTKHPHLMGSSVLGMNDIYRTWRAFVLRVRALDQTPRMYFVKAAITGAYDAIPQGKLVEVVANMIRHSESTYCIRQYAVVRRDSQGQVHKSFRRQVTTLSDLQPYMGQFLKHLQDSDASALRNSVVIEQSISMNESSSSLFDFFLHFLRHSVVKIGDRCYTQCQGIPQGSSLSTLLCSLCFGDMENKLFAEVQRDGLLLRFVDDFLLVTPHLDQAKTFLSTLVHGVPEYGCMINLQKTVVNFPVEPGTLGGAAPYQLPAHCLFPWCGLLLDTQTLEVFCDYSGYAQTSIKTSLTFQSVFKAGKTMRNKLLSVLRLKCHGLFLDLQVNSLQTVCINIYKIFLLQAYRFHACVIQLPFDQRVRKNLTFFLGIISSQASCCYAILKVKNPGMTLKASGSFPPEAAHWLCYQAFLLKLAAHSVIYKCLLGPLRTAQKLLCRKLPEATMTILKAAADPALSTDFQTILDSRAPQSITELCSEYRNTQIYTINDKILSYTESMAGKREMVIITFKSGATFQVEVPGSQHIDSQKKAIERMKDTLRITYLTETKIDKLCVWNNKTPNSIAAISMEN

SEQ ID NO: 11 - protein, 253 aa, Artificial Sequence
  Description of Artificial Sequence Synthetic polypeptide
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKLRGSHHHHHHAAAS

SEQ ID NO: 12 - DNA, 765 bp, Artificial Sequence
  Description of Artificial Sequence Synthetic polynucleotide
catatggtgtccaaaggcgaagaactgttcaccggcgtggtgccgattctggtggaactggatggcgatgtgaacggccacaaattcagcgtgtccggcgaaggtgaaggtgatgccacctacggcaaactgaccctgaaattcatctgtaccaccggcaaactgccggtgccgtggccgaccctggtgaccaccctgacctacggcgtgcagtgcttctctcgctacccggatcacatgaaacagcacgatttcttcaaaagcgccatgccggaaggctacgtgcaggaacgtaccattttcttcaaagatgatggcaactacaaaacccgtgccgaagtgaaattcgaaggcgataccctggtgaaccgtatcgaactgaaaggcatcgactttaaagaggacggtaacatcctgggccacaaactggaatacaactacaacagccacaacgtgtacatcatggccgataaacagaaaaacggcatcaaagtgaacttcaaaatccgccacaacatcgaagatggcagcgtgcagctggccgatcactaccagcagaacaccccgattggtgatggcccggtgctgctgccggataaccactacctgagcacccagagcgccctgagcaaagatccgaacgaaaaacgtgatcacatggtgctgctggaattcgtgaccgccgctggtattaccctgggcatggatgaactgtacaagcttagaggatctcaccatcaccatcaccatgcggccgcatcgtga

SEQ ID NO: 13 - DNA, 765 bp, Artificial Sequence
  Description of Artificial Sequence Synthetic polynucleotide
catatggtgagtaaaggtgaagaattattcacgggcgtggttccaattctggttgaactggatggcgatgtgaacggtcacaaattcagtgttagcggcgaaggcgaaggtgatgcgacgtacggcaaactgacgctgaaattcatctgtaccaccggcaaactgccggttccatggccgacgctggttacgaccttaacctacggcgttcagtgcttcagtcgttacccagatcacatgaaacagcacgatttcttcaaaagcgccatgccagaaggttacgttcaggaacgtacgattttcttcaaagatgatggcaactacaaaacccgtgcggaagtgaaattcgaaggtgataccttagtgaaccgtatcgaattaaaaggcatcgactttaaagaggacggcaacatcttaggtcacaaattagaatacaactacaacagccacaacgtgtacatcatggcggataaacagaaaaacggcatcaaagttaacttcaaaatccgccacaacatcgaagatggtagtgtgcagttagcggatcactaccagcagaacaccccgattggcgatggcccggttttactgccagataaccactacctgagtacccagagtgccctgagcaaagatccaaacgaaaaacgtgatcacatggttttactggaattcgttacggcggcgggcattacgctgggcatggatgaactgtacaagcttagaggatctcaccatcaccatcaccatgcggccgcatcgtga

SEQ ID NO: 14 - protein, 250 aa, Artificial Sequence
  Description of Artificial Sequence Synthetic polypeptide
MAAPSDGFKPRERSGGEQAQDWDALPPKRPRLGAGNKIGGRRLIVVLEGASLETVKVGKTYELLNCDKHKSILLKNGRDPGEARPDITHQSLLMLMDSPLNRAGLLQVYIHTQKNVLIEVNPQTRIPRTFDRFCGLMVQLLHKLSVRAADGPQKLLKVIKNPVSDHFPVGCMKVGTSFSIPVVSDVRELVPSSDPIVFVVGAFAHGKVSVEYTEKMVSISNYPLSAALTCAKLTTAFEEVWGVIHHHHHH

SEQ ID NO: 15 - DNA, 764 bp, Artificial Sequence
  Description of Artificial Sequence Synthetic polynucleotide
ccatggctgctcctagcgacggcttcaagccccgggagcggagcggcggagagcaggcccaggactgggacgccctgccccccaagcggcctagactgggagccggcaacaagatcggcggcaggcggctgatcgtggtgctggaaggcgccagcctggaaaccgtgaaagtgggcaagacctacgagctgctgaactgcgacaagcacaagagcatcctgctgaagaacggccgggaccccggcgaggccaggcccgacatcacccaccagagcctgctgatgctcatggattcccccctgaacagagccggcctgctgcaggtgtacatccacacccagaaaaacgtgctgatcgaggtgaacccccagaccagaatcccccggaccttcgaccggttctgcggcctgatggtccagctgctccataagctgtccgtgagagccgccgacggcccccagaaactgctgaaggtgatcaagaaccccgtgagcgaccacttccccgtgggctgcatgaaagtggggaccagcttcagcatccccgtggtgtccgacgtgcgggagctggtgcccagcagcgaccccatcgtgttcgtggtgggcgccttcgcccacggcaaggtgtccgtggagtacaccgagaagatggtgtccatcagcaactaccccctgtctgccgccctgacctgcgccaagctgaccaccgccttcgaggaagtgtggggcgtgatccaccaccaccaccaccactgataactcgag

SEQ ID NO: 16 - DNA, 764 bp, Artificial Sequence
  Description of Artificial Sequence Synthetic polynucleotide
ccatggccgctcctagcgacggcttcaagcccagagagcgctccggcggagagcaggcccaggactgggacgccctcccccccaagagacctagactcggagccggaaacaagatcggcggcaggaggctcatcgtcgtgctggaaggcgcttccctggaaacagtgaaagtgggaaagacctacgagttgctcaactgcgacaagcacaagtccatcctcctcaagaacggaagggaccctggcgaggctaggcctgacatcacacaccagagcctgctcatgctcatggatagccccctgaacagggctggactcctccaggtctacatccacacccagaaaaacgtgctcatcgaggtcaaccctcagacaagaatccctaggacattcgacaggttctgcggcctgatggtgcagctcctgcataagctctccgtcagggctgctgacggacctcagaaactgctgaaggtcatcaagaaccccgtcagcgaccacttccccgtgggatgcatgaaagtcggcacctcattcagcatccctgtcgtcagcgacgtcagagagttggtcccctcctccgaccccatcgtcttcgtcgtgggcgctttcgcccacggaaaggtgtccgtcgagtacacagagaagatggtgtccatcagcaactaccctctgtccgccgctctgacctgcgctaagctcaccacagccttcgaggaagtgtggggcgtgatccaccaccaccaccaccactgataactcgag

SEQ ID NO: 17 - DNA, 764 bp, Artificial Sequence
  Description of Artificial Sequence Synthetic polynucleotide
ccatggctgccccctccgacggcttcaagcctagagagaggagcggaggggagcaggctcaggactgggacgccctgcctcctaagaggcccagactgggagccggcaacaagatcggcggcaggaggctgatcgttgtcctcgaaggagctagcctggaaacagtgaaagtcggaaagacctacgagctgctgaactgcgacaagcacaagtccatcctcctcaagaacggcagggaccccggcgaggctaggcccgacatcacacaccagtccctgctgatgctgatggattcccctctgaacagggctggactgctccaggtgtacatccacacacagaaaaacgtcctcatcgaggttaaccctcagacaaggatccccaggaccttcgacaggttctgcggactgatggtgcagctgctccataagctcagcgtcagggctgctgacggcccccagaaactcctcaaagtcatcaagaaccccgttagcgaccacttccccgtgggctgcatgaaagtcggaacaagcttctccatccctgttgtcagcgacgtcagggagttggtgcctagctccgaccccatcgtgttcgtcgtcggagctttcgcccacggaaaagttagcgtggagtacaccgagaagatggtctccatcagcaactaccccctgtccgcagccctcacctgcgccaagctgacaaccgctttcgaggaagtgtggggcgtgatccaccaccaccaccaccactgataactcgag

SEQ ID NO: 18 - protein, 6 aa, Artificial Sequence
  Description of Artificial Sequence Synthetic 6xHis tag
HHHHHH

SEQ ID NO: 19 - protein, 5 aa, Artificial Sequence
  Description of Artificial Sequence Synthetic peptide
VPTAG

SEQ ID NO: 20 - protein, 10 aa, Artificial Sequence
  Description of Artificial Sequence Synthetic peptide
DEKNIQHCYF

SEQ ID NO: 21 - protein, 18 aa, Artificial Sequence
  Description of Artificial Sequence Synthetic peptide
GEDAVRSKNTIQHPLCYF

(19) United States
(12) Patent Application Publication
(10) Pub. No.: US 2015/0125949 A1
(43) Pub. Date: May 7, 2015

(54) STEGANOGRAPHIC EMBEDDING OF INFORMATION IN CODING GENES

(71) Applicant: GENEART AG, Carlsbad, CA (US)

(72) Inventor: Michael LISS, Regensburg (DE)

(21) Appl. No.: 14/340,550

(22) Filed: Jul. 24, 2014

Related U.S. Application Data
(62) Division of application No. 12/745,204, filed on Dec. 14, 2010, now abandoned.

(30) Foreign Application Priority Data
Nov. 30, 2007 (DE) 102007057802.6

(51) Int. Cl. C07H 1/00; C12N 15/63; H04L 9/08; C07H 21/04

(52) U.S. Cl. CPC C07H 1/00; C07H 21/04; C12N 15/63; H04L 9/0816; H04L 2209/24
USPC 435/320.1; 536/23.1

(57) ABSTRACT

The present invention relates to the storage of items of information in nucleic acid sequences. The invention also relates to nucleic acid sequences in which desired items of information are contained, and to the design, production or use of such sequences.

The present invention relates to the storage of information in nucleic acid sequences. The invention furthermore relates to nucleic acid sequences which contain desired information, and to the design, production or use of such sequences.

Important information, especially secret information, must be protected from unauthorised access. Ever more elaborate cryptographic or steganographic techniques have in the past been developed for this purpose. There are numerous algorithms in existence for encrypting data and for camouflaging secret information. The security of an item of secret steganographic information depends, among other things, on its existence not being obvious to an unauthorised person. The information is packaged in an unobtrusive medium, it being in principle possible to select the medium at will. For example, it is known in the prior art to conceal information in digital images or audio files. One pixel of a digital RGB image consists of 3×8 bits. Each 8 bits encode the brightness of the red, green and blue channels respectively. Each channel can accommodate 256 brightness levels. If the last bit (least significant bit, LSB) of each pixel and channel is overwritten with an item of foreign information, the brightness of each channel changes by only 1/256, thus by 0.4%. To an observer the image remains unchanged in appearance.

Music on a CD is digitised at 44,100 samples/second, 2 channels, 16 bits/sample. Overwriting the LSB of a sample changes the wave amplitude at this point by 1/65536, thus by 0.002%. This change is not audible to humans. A conventional CD thus offers space for 74 min×60 sec×44,100 samples×2 channels=392 Mbits or approx. 50 Mbytes.

Recent years have moreover seen the development of steganographic approaches based on DNA. Clelland et al. (Nature 399:533-534 and U.S. Pat. No. 6,312,911), inspired by the microdots used in the second world war, developed a method for concealing messages in “DNA microdots”. They produced artificial DNA strands which were assembled from a series of triplets, to each of which was assigned a letter or number. In order to decode the message, the recipient of the secret information must know the primers for amplification and sequencing and the decryption code.

U.S. Pat. No. 6,537,747 discloses methods for encrypting information from words, numbers or graphic images. The information is directly incorporated into nucleic acid strands which are sent to the recipient who can decode the information using a key.

The methods described by Clelland and in U.S. Pat. No. 6,537,747 are in each case based on the direct storage of information in DNA. However, the disadvantage of such direct storage by a simple triplet code is that conspicuous sequence motifs may arise which could be noticed by third parties. As soon as it has been recognised that a medium contains an item of secret information, there is a risk that this information will also be decrypted. Furthermore, such DNA domains can perform a biologically relevant function only to a very limited extent. When producing genetically modified organisms, the nucleic acids which contain the encrypted message must accordingly be introduced in addition to the genes which bring about the desired characteristics of the organism.

It was accordingly the object of the present invention to provide an improved steganographic method for embedding information in nucleic acids which is more secure from unwanted decryption. The intention is to conceal the information in such a manner that a third party cannot even recognise that it contains an item of secret information.

The inventors of the present invention have found out that the degeneracy of the genetic code can be exploited in order to embed information in coding nucleic acids. The degeneracy of the genetic code is taken to mean that a specific amino acid can be encoded by different codons. A codon is defined as a sequence of three nucleobases which encodes an amino acid in the genetic code. According to the invention, a method has been developed with which nucleic acid sequences are provided which are modified in such a manner that they contain a desired item of information.

In a first aspect, the present invention provides a method for designing nucleic acid sequences containing information which comprises the steps:
(a) assigning a first specific value to at least one first nucleic acid codon from a group of degenerate nucleic acid codons which encode the same amino acid,
   assigning a second specific value to at least one second nucleic acid codon from the group,
   optionally assigning one or more further specific values to in each case at least one further nucleic acid codon from the group,
   in which the first and second and optionally further values within the group of codons which encode the same amino acid are in each case allocated at least once;
(b) providing an item of information to be stored as a series of n values which are in each case selected from first and second and optionally further values, in which n is an integer ≧1;
(c) providing a starting nucleic acid sequence, the sequence comprising n degenerate codons to which are assigned according to (a) first and second and optionally further values, in which n is an integer ≧1; and
(d) designing a modified sequence of the nucleic acid from (c), in which, at the positions of the n degenerate codons of the starting nucleic acid sequence, in each case one nucleic acid codon is selected from the group of degenerate codons which encode the same amino acid, which codon, by the assignment from (a), corresponds to a value such that the series of the values assigned to the n codons gives rise to the information to be stored.

There are in total 64 different codons available in the genetic code which encode in total 20 different amino acids and stop. (Stop codons are in principle also suitable for accommodating information.) A plurality of codons is accordingly used for many amino acids and for stop. For example, the amino acids Tyr, Phe, Cys, Asn, Asp, Gin, Glu, His and Lys are in each case two-fold encoded. There are in each case three degenerate codons for the amino acid Ile and for stop. The amino acids Gly, Ala, Val, Thr and Pro are in each case four-fold encoded and the amino acids Leu, Ser and Arg are in each case six-fold encoded. The different codons which encode the same amino acid generally differ in only one of the three bases. Usually, the codons in question differ in the third base of a codon.

Step (a) of the method according to the invention exploits this degeneracy of the genetic code in order to assign specific values to degenerate nucleic acid codons within a group of codons which encode the same amino acid. In step (a), within a group of degenerate nucleic acid codons which encode the same amino acid, a first specific value is assigned to at least one first nucleic acid codon and a second specific value is assigned to at least one second nucleic acid codon from this group. The first and second values within the group of codons which encode the same amino acid are here in each case allocated at least once.

This assignment may be made for one or more of the multiply-encoded amino acids. In principle, such an assignment may be made for all multiply-encoded amino acids. Preferably, an assignment is only made for the at least three-fold, preferably at least four-fold, more preferably six-fold encoded amino acids. It is particularly preferred according to the invention to assign specific values only to the codons of four-fold encoded amino acids and/or to the codons of the six-fold encoded amino acids.

If also the two-fold encoded amino acids are included in the assignment in step (a), only a first and a second value may be assigned. If only the at least four-fold encoded amino acids are included, in total up to four different values may be allocated within a group of degenerate nucleic acid codons which encode the same amino acid. If only six-fold encoded amino acids are included, up to six different values may accordingly be allocated within a group of degenerate nucleic acid codons.

By the assignment of more than two, i.e. in particular of four or six different values within a group, it is possible to store a larger volume of information by means of a shorter series of codons. One embodiment according to the invention accordingly provides assigning values in step (a) only to the codons of those amino acids which are at least four-fold, preferably six-fold encoded. Within the group of degenerate nucleic acid codons which encode the same multiply-encoded amino acid, first and second and one or more further values are then preferably assigned to in each case at least one nucleic acid codon from the group. The first and second and optionally further values are in each case allocated at least once within the group of codons.

If only the at least four-fold or six-fold encoded amino acids are included in the assignment of step (a), it is alternatively also possible, within a group of degenerate nucleic acid codons which encode the same amino acid, to assign a first specific value to more than one first nucleic acid codon, i.e. two, three, four or five nucleic acid codons, and/or to assign a second specific value to more than one second nucleic acid codon from the group, i.e. two, three, four or five nucleic acid codons. Preferably, the first and second values within the group of degenerate codons are in each case allocated repeatedly, preferably equally often. Within a group of degenerate nucleic acid codons which encode the same four-fold encoded amino acid, this means that preferably a first value is assigned to two nucleic acid codons and a second value is assigned to two other codons. Correspondingly, if six-fold encoded amino acids are included, a first value is preferably assigned to three nucleic acid codons from a group and a second value is assigned to three other nucleic acid codons which encode the same amino acid. In this manner, at least two possible codons which encode the same amino acid are available for each first and for each second value. The alternative of several possible codons for one specific value makes it possible to avoid unwanted sequence motifs.

In a preferred embodiment of the invention, in step (a) a specific value is assigned to all the nucleic acid codons from a group of degenerate nucleic acid codons which encode the same amino acid. It is, however, also possible according to the invention to assign a value to only individual ones of the degenerate nucleic acid codons and not to take account of other nucleic acid codons which encode the same amino acid.

In step (b) of the method according to the invention, an item of information to be stored is provided as a series of n values which are in each case selected from first and second and optionally further values, n here being an integer ≧1. The information to be stored may, for example, comprise graphic, text or image data. The information to be stored may be provided as a series of n values in step (b) in any desired manner. Care must be taken to select the n values from the same first and second and optionally further values which are assigned to specific nucleic acid codons in step (a). Thus, if for example only first and second values are assigned in step (a), the information to be stored in step (b) must be provided as a series of values which are selected from said first and second values. The information to be stored is accordingly provided in binary form. To this end, text data for example may be represented in binary form by means of the ASCII code, which is known in the field. If in step (a), in addition to the first and second values, one or more further values are also assigned, the information to be stored may be provided in step (b) as a series of n values which are selected from first and second and these further values.

In a preferred embodiment, the information to be stored is not directly converted into a series of n values, but instead previously encrypted in any desired known manner. Only once it is encrypted is the information then converted into a series of n values as described above. Encryption algorithms usable for this purpose are known in the prior art, such as for example the Caesar cipher, Data Encryption Standard, one-time pad, Vigenere, Rijndael, Twofish, 3DES. (Literature regarding encryption algorithms: Bruce Schneier: Applied Cryptography, John Wiley & Sons, 1996, ISBN 0-471-1109-9).

A starting nucleic acid sequence is provided in step (c) of the method according to the invention. The starting nucleic acid sequence may be selected at will. For example, the nucleic acid sequence of a naturally occurring polynucleotide may be used. According to the invention, “polynucleotide” is taken to mean an oligomer or polymer made up of a plurality of nucleotides. The length of the sequence is not in any way limited by the use of the term polynucleotide, but instead according to the invention comprises any desired number of nucleotide units. The starting nucleic acid sequence is, according to the invention, particularly preferably selected from RNA and DNA. The starting nucleic acid may, for example, be a coding or non-coding DNA strand. The starting nucleic acid sequence is particularly preferably a naturally occurring coding DNA sequence which encodes a specific protein.

The starting nucleic acid sequence comprises n degenerate codons, to which are assigned first and second and optionally further values according to (a), n is an integer ≧1 and corresponds to the number of n values of the information to be stored from step (b). The n degenerate codons may alternatively be arranged in immediate succession in the starting nucleic acid sequence or their series may be interrupted by other non-degenerate codons or degenerate codons to which no value is assigned according to (a). It is moreover possible for the series of n degenerate codons to be interrupted at one or more points by non-coding domains. In a preferred embodiment, the n degenerate codons are present in an uninterrupted coding sequence. The starting nucleic acid particularly preferably encodes a specific polypeptide.

A modified sequence of the nucleic acid sequence from (c) is designed in step (d) of the method according to the invention. In the modified sequence, at the positions of the n degenerate codons of the starting nucleic acid sequence, nucleic acid codons from the group of degenerate codons which encode the same amino acid are in each case selected, to which a value has been assigned by the assignment from (a). The degenerate codons are selected such that the series of the values assigned to the n codons gives rise to the information to be stored.

If the starting nucleic acid sequence encodes a polypeptide, the modified sequence designed in step (d) preferably encodes the same polypeptide. According to the invention, “polypeptide” is taken to mean an amino acid chain of any desired length.

In one embodiment according to the invention, the start and/or end of an item of information in the modified sequence from step (d) may be marked by incorporating an agreed stop sign. For example, the series of n codons which gives rise to the information to be stored may be followed by a series of two or more codons to which the same value is assigned.

In one particularly preferred embodiment, in step (a) a first or second or optionally further value is assigned to a nucleic acid codon within the group of degenerate codons which encode the same amino acid, depending on the frequency with which the codon is used in a specific organism. Different values may be assigned to various degenerate codons on the basis of a species-specific codon usage table (CUT). For example, within a group of degenerate nucleic acid codons which encode the same amino acid, a first value may be assigned to the first best codon, i.e. to the codon most frequently used by a species, and a second value to a second best codon. If only the at least four-fold or six-fold coded amino acids are included in the assignment of step (a), one or more further values within the group of degenerate codons which encode the same amino acid may be allocated in this manner. In a preferred embodiment, only first and second values within the group are allocated.

For example, in one embodiment, a first value is assigned to the first and the third best codon while a second value is assigned to the second and the fourth best codon. Any desired types of assignment are possible according to the invention, providing that at least one first and at least one second value is assigned within a group of degenerate codons which encode the same amino acid.

By the alternative of two or more possible codons per value within a group of degenerate codons it is possible, when designing a modified sequence-in step (d), to avoid unwanted sequence motifs.

If two or more codons have the same frequency in a species-specific codon usage table, a further condition is agreed upon for the assignment of values.

As an alternative to the assignment of values on the basis of the frequency of use of a codon within a group of degenerate codons or as a further condition, as mentioned above, assignment may also be made on the basis of alphabetic sorting. Numerous further options for assignment are furthermore conceivable and the present invention is not intended to be limited to assignment based on the frequency of codon use.

In one particularly preferred embodiment of the method according to the invention, the modified nucleic acid sequence designed in step (d) may be produced in a subsequent step (e). Production may proceed by any desired method known in the field. For example, a nucleic acid with the modified sequence designed in step (d) may be produced from the starting sequence of step (c) by mutation. In particular, substitution of individual nucleobases is suitable for this purpose. Mutation by insertions and deletions is likewise possible. A nucleic acid with the modified sequence may moreover be produced synthetically in step (e). Methods for producing synthetic nucleic acids are known to a person skilled in the art. The method according to the invention gives rise to a modified nucleic acid sequence which contains a desired item of information in encrypted form. Its key resides in the assignment of step (a). This key must be known to an addressee of the information. For example, the key can be sent separately to the addressee at a different time.

In one particularly preferred embodiment, the key for the assignment according to (a) may itself be encrypted and stored in a nucleic acid. For example, the key may additionally be incorporated into the modified nucleic acid sequence obtained in the method according to the invention or be separately incorporated into another nucleic acid. The key for the assignment of (a) is generally encrypted using another key. Known prior art methods may in principle be used for this purpose. So that the key deposited in a nucleic acid may be found, it is preferably accommodated at an agreed location, for example immediately downstream of a stop codon, downstream of the 3′ cloning site or the like, it may also be accommodated at an entirely different location within the genome or episomally. By flanking the key sequence with specific primer binding sites (known only to the initiated), this key is then only accessible via a specific PCR and sequencing the PCR product. It is moreover advantageous also to encrypt the deposited key sequence itself with a password so that it is not recognisable as such. Encryption algorithms usable for this purpose are known in the prior art, for example Caesar cipher, Data Encryption Standard, one-time pad, Vigenère, Rijndael, Twofish, 3DES. (Literature regarding encryption algorithms: Bruce Schneier: Applied Cryptography, John Wiley & Sons, 1996, ISBN 0-471-11709-9).

The present invention furthermore comprises a modified nucleic acid sequence which is obtainable by a method according to the invention, and a modified nucleic acid which comprises this nucleic acid sequence and may be obtained using the method according to the invention. Methods for producing nucleic acids are known to a person skilled in the art. Production may, for example, proceed on the basis of phosphoramidite chemistry, by chip-based synthesis methods or solid phase synthesis methods. It goes without saying that any desired other synthesis methods which are familiar to a person skilled in the art may furthermore also be used.

The present invention furthermore provides a vector which comprises a nucleic acid modified according to the invention. Methods for inserting nucleic acids into any desired suitable vector are known to a person skilled in the art.

The invention furthermore relates to a cell which comprises a nucleic acid modified according to the invention or a vector according to the invention, and to an organism which comprises a nucleic acid or cell according to the invention or a vector according to the invention.

In a further embodiment, the present invention relates to a method for sending a desired item of information, in which a nucleic acid sequence according to the invention, a nucleic acid, a vector, a cell and/or an organism is sent to a desired recipient. Before being sent to the recipient, it is particularly preferred to mix the nucleic acid, the vector, the cell or the organism with other nucleic acids, vectors, cells or organisms which do not contain the desired information. These “dummies” may, for example, contain no information or contain other information acting as a diversion and not representing the desired information.

Moreover, the information contained in a nucleic acid sequence modified according to the invention may also act as a “watermark” for marking a gene, a cell or an organism. The present invention accordingly provides in one embodiment the use of a nucleic acid sequence modified according to the invention for marking a gene, a cell and/or an organism. Marking genes, cells or organisms with a watermark according to the invention allows them to be definitely identified. Origin and authenticity may accordingly be definitely established. A gene, a cell or an organism is marked with a “watermark” according to the invention by modifying a natural nucleic acid sequence of the gene or of the cell or of the organism or part of the sequence as described above. At the positions of degenerate codons of the starting sequence, codons which encode the same amino acid (or likewise stop) are in each case selected to which a specific value has been assigned. The codons are selected such that the series of the values assigned thereto in the nucleic acid sequence corresponds to a specific characteristic. This marking cannot be recognised by a third party; functioning of the gene, cell or organism is not impaired.

The following Figures and examples further illustrate the invention.

### FIGURES

FIG. 1: Extract from the international ASCII table.

FIGS. 2A-2B: shows the test gene used in Example 1 (mouse telomerase), optimised for H. sapiens (A) and the encoded protein (B)

FIG. 3: Codon usage table (CUT) for Homo sapiens

FIG. 4: Codon order of the permutations

FIG. 5 shows an analysis of the modified sequence obtained in Example 1 in comparison with the starting sequence FIG. 6 shows an alignment of the sequences of eGFP(opt) and eGFP(msg) from Example 3. The translated amino acid sequence of the protein eGFP is shown above the alignment. Silent substitutions arising from the use of alternative codons on embedding the message “AEQUOREA VICTORIA.” in eGFP(msg) are highlighted in black. Cloning sites are underlined, the vector content of the 6×His-tag is also shown downstream of the 3′ HindIII restriction site.

FIG. 7 shows the results of analysis of the expression of the genes eGFP(opt) and eGFP(msg) from Example 3 by Coomassie gel, Western blot (with a GFP-specific antibody) and fluorescence analysis:

FIG. 8 shows an alignment of the sequences of EMG1(opt), EMG1 (msg) and EMG1 (enc) from Example 4. The translated amino acid sequence of the protein EMG1 is shown above the alignment. Silent substitutions arising from the use of alternative codons on embedding the message “GENEARTAG PAT U.S. Pat. No. 1,234,567” in EMG1(msg) and the encrypted message “:JQWF&G % DY %$4Y#′XE %87G;K” in EMG1 (enc) are highlighted in black. Cloning sites are underlined.

FIG. 9 shows the result of the analysis of the expression of EMG 1(opt), EMG1(msg) and EMG1 (enc) by means of Western blot analysis using a His-specific antibody.

### EXAMPLES

### Example 1

### Encryption of “GENE” in the N Terminus of M. musculus Telomerase (Optimised for H. sapiens)

The N terminus of M. musculus telomerase was selected as the medium for encrypting the message “GENE”. M. musculus telomerase (1251AA) comprises 360 four-fold degenerate, information-containing codons (ICCs) and 372 six-fold degenerate ICCs. The open reading frame (ORF) of the gene is first of all optimised in conventional manner, i.e. codon selection is adapted to the specific circumstances of the target organism.

Below, consideration is given only to those codons which are 4- and 6-fold degenerate, thus for the amino acids VPTAG (each 4 codons) and LSR (each 6 codons). These are designated ICC (information containing codons). (Amino acids for which there are only 2 or 3 codons (DEKNIQHCYF) may in principle also be used, but since gene performance suffers more severely, they are disregarded in the present example.)

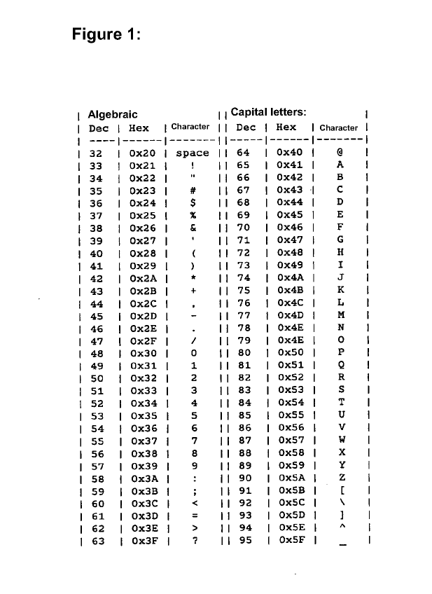

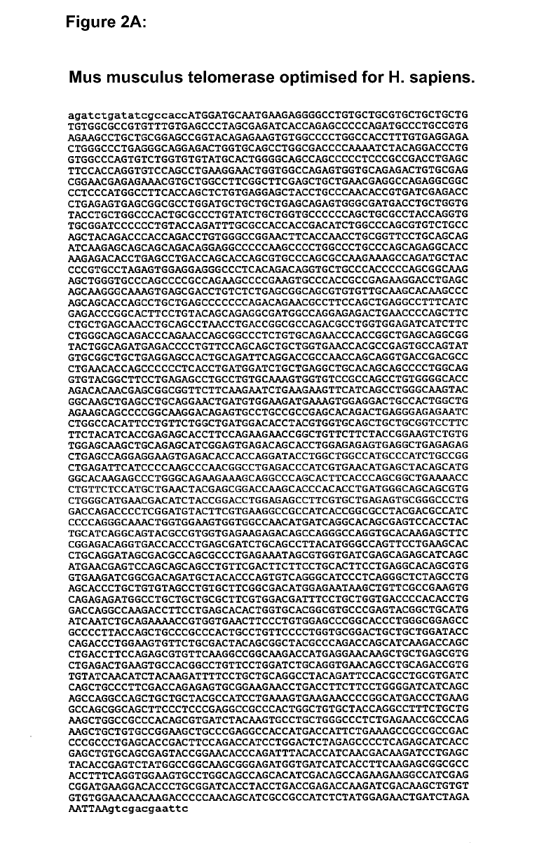

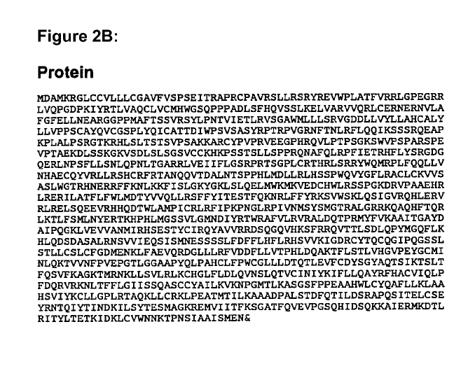

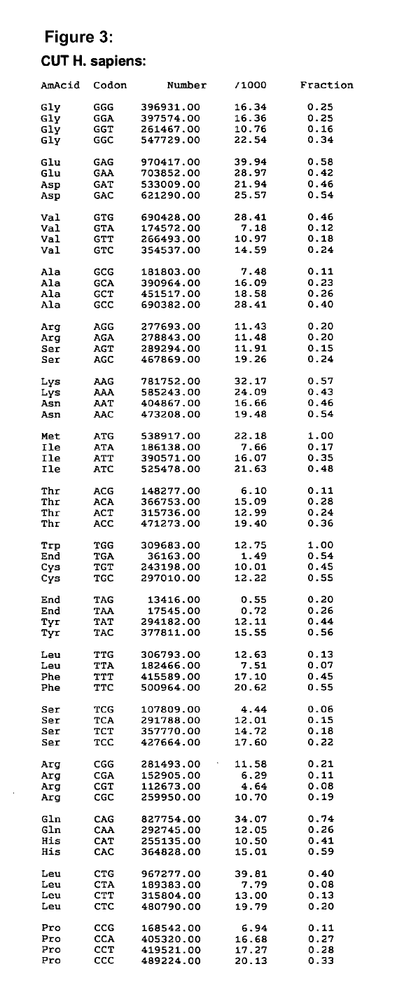

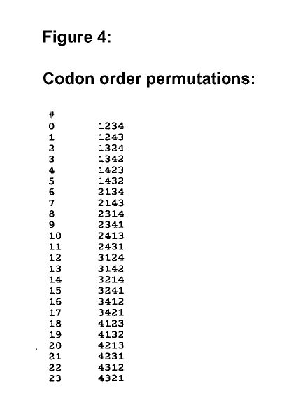

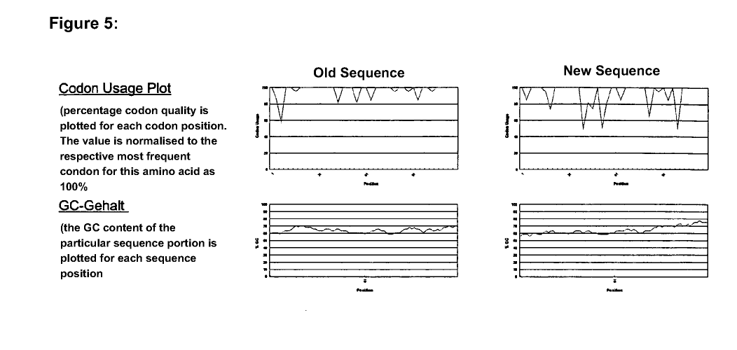

The secret information (under certain circumstances previously encrypted) is now broken down into bits. 6 bits (=2⁶=64 states) Der character are here sufficient for letters+numbers+special characters; ideally the ASCII characters from 32=0010 0000 (space) to 95=0101 1111 (underscore). This range includes capital letters, numbers and the most important special characters (see FIG. 1), The eight digit ASCII code is reduced to a 6 bit code using the conventional bit operation: 6 bits=8 bits−32 or 8 bits=6 bits+32.

The CUT below for Homo sapiens is used for encryption in this example:

ICC CUT H. sapiens (sorted by “fraction” (1) & alphabetically (2))

| AA | Codon | Fraction | AA | Codon | Fraction | AA | Codon | Fraction | AA | Codon | Fraction |
|---|---|---|---|---|---|---|---|---|---|---|---|
| A | GCC | 0.40 | P | CCC | 0.33 | V | GTG | 0.46 | R | CGG | 0.21 |
| A | GCT | 0.26 | P | CCT | 0.28 | V | GTC | 0.24 | R | AGA | 0.20 |
| A | GCA | 0.23 | P | CCA | 0.27 | V | GTT | 0.18 | R | AGG | 0.20 |
| A | GCG | 0.11 | P | CCG | 0.11 | V | GTA | 0.12 | R | CGC | 0.19 |
| G | GGC | 0.34 | T | ACC | 0.36 | L | CTG | 0.40 | R | CGA | 0.11 |
| G | GGA | 0.25 | T | ACA | 0.28 | L | CTC | 0.20 | R | CGT | 0.08 |
| G | GGG | 0.25 | T | ACT | 0.24 | L | CTT | 0.13 | S | AGC | 0.24 |
| G | GGT | 0.16 | T | ACC | 0.11 | L | TTG | 0.13 | S | TCC | 0.22 |
| | | | | | | L | CTA | 0.08 | S | TCT | 0.18 |
| | | | | | | L | TTA | 0.07 | S | AGT | 0.15 |
| | | | | | | | | | S | TCA | 0.15 |
| | | | | | | | | | S | TCG | 0.06 |

On the basis of the species-specific codon usage table (CUT), all ICCs from 5′ to 3′ are successively modified and the additional information introduced bit by bit. The following applies:

Binary 1=first or third best codon
Binary 0=second or fourth best codon

The “first best”-“fourth best” codon weighting here reflects the frequency with which the respective codon is used in the target organism for encoding its amino acid. A database on this subject may be found at: http://www.kazusa.or.jp/codon/.

The alternative of two possible codons per bit makes it possible, most probably in every case, to avoid unwanted sequence motifs duting optimisation. ICC-adjacent non-ICC codons may, of course, also be modified in order to exclude specific motifs.

A defined CUT is necessary for definite encryption and decryption. However, especially for little investigated organisms, CUTs will still change in future. It is therefore necessary in many cases to deposit a dated CUT. However, only the order of the ICC codons is of relevance, not the actual frequency figures.

The order may be deposited on paper or notarially. It is, of course, possible also to accommodate these data in the DNA itself, for example the 3′ UTR (immediately downstream from the gene). 22 nt are required for deposition of the ICC CUT (see Example 2).

However, for the commonest target organisms (mammals, crop plants, E. coli, baker's yeast etc.), the codon tables are so complete that they will not change any further.

If two or more codons have the same frequency in the CUT, the codons in question are sorted alphabetically: A>C>G>T.

The end of a message may be marked with an agreed stop character, for example “11 1111”, corresponding to the underscore character.

The strategy of defining the first or third best codon as binary 1 and the second or fourth best codon as binary 0, i.e. in general of working with a codon usage table, gives rise to a gene which is firstly largely optimised and thus functions well in the target organism and secondly permits a watermark.

Alternatively, it is in principle also possible to define all amino acids for which there are two or more codons as ICC and to agree on the following coding principle for steganographic data embedding:

Binary 1=G or C at codon position 3
Binary 0=A or T at codon position 3

This is possible for the 18 amino acids GEDAVRSKNTIQHPLCYF. (In the above method based on a quality ranking, there are only 8 ICCs.) In this manner, more than twice as much information may be accommodated in a gene and a definite CUT need not be deposited in any case. The disadvantage of this method is, however, that the resultant gene is not optimised or is scarcely so.

In the present example, the message “GENE” was encrypted in the N terminus of M. musculus telomerase. This message contains 4×6=24 bits.

| | G | E | N | E |
|---|---|---|---|---|
| “GENE”, binary 8 bit: | 0100 0111 | 0100 0101 | 0100 1110 | 0100 0101 |
| | (71) | (69) | (78) | (69) |
| 8 bit - 32: | (39) | (37) | (46) | (37) |
| “GENE”, binary 6 bit: | 10 0111 | 10 0101 | 10 1110 | 10 0101 |

24 bits were encrypted by modifying 10 four-fold or six-fold deqenerate ICCs in the N terminus of the telomerase:

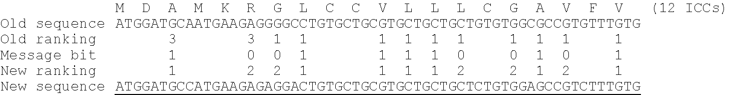

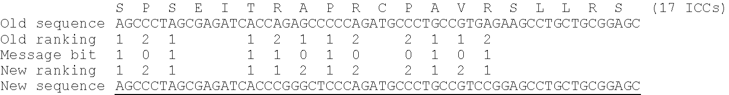

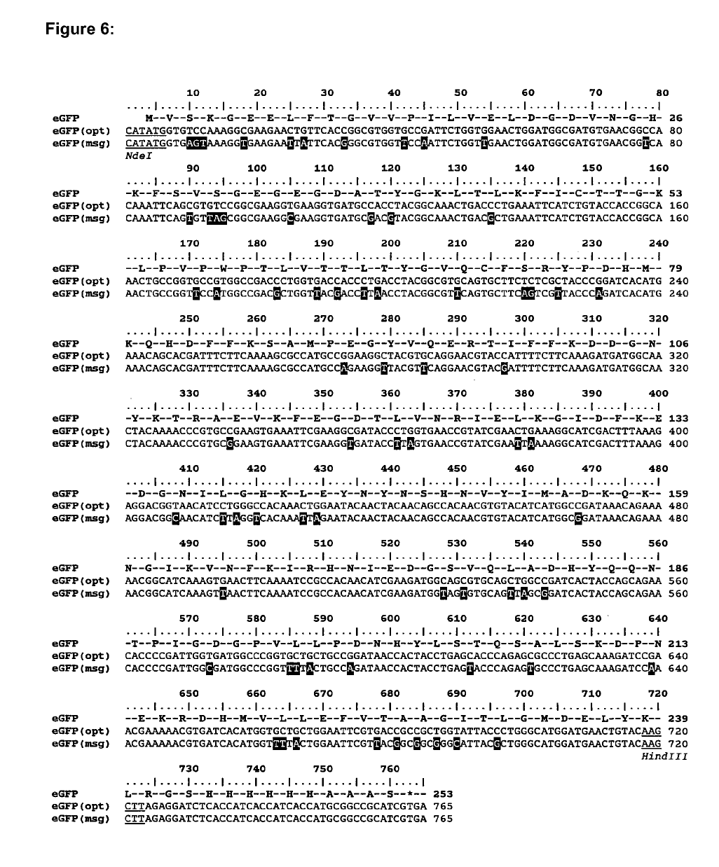

No unwanted motifs nor an excessively high GC content occurred during coding. It was therefore not necessary to make use of the third best and fourth best codons. FIG. 5 shows a comparison of the analysis of the starting sequence and of the modified sequence.

### Example 2

### Encryption of the Codon Usage Table for Escherichia coli and Deposition as a Nucleic Acid Sequence

It is essential to know the coding used in order to encrypt the information embedded in the genes. It is the key for decoding and may preferably consist of the codon usage table predetermined by the organism. In principle, however, the key used may be selected at will from approx. 5.48×10¹⁹ possible combinations.

It is possible likewise to encode this key in the form of a specific nucleotide sequence and so deposit it, for example, within the genome.

The codon usage table is firstly sorted alphabetically by amino acid and then the codons of an amino acid are sorted alphabetically by codon:

| Amino acid | Codon | Frequency | Rank |
|---|---|---|---|
| A | GCA | 0.22 | 3 |
| A | GCC | 0.27 | 2 |
| A | GCG | 0.35 | 1 |
| A | GCT | 0.16 | 4 |
| C | TGC | 0.55 | 1 |
| C | TGT | 0.45 | 2 |
| D | GAC | 0.37 | 2 |
| D | GAT | 0.63 | 1 |
| E | GAA | 0.68 | 1 |
| E | GAG | 0.32 | 2 |
| F | TTC | 0.42 | 2 |
| F | TTT | 0.58 | 1 |
| G | GGA | 0.12 | 4 |
| G | GGC | 0.38 | 1 |
| G | GGG | 0.16 | 3 |
| G | GGT | 0.33 | 2 |
| H | CAC | 0.42 | 2 |
| H | CAT | 0.58 | 1 |
| I | ATA | 0.09 | 3 |
| I | ATC | 0.40 | 2 |
| I | ATT | 0.50 | 1 |
| K | AAA | 0.16 | 1 |
| K | AAG | 0.24 | 2 |
| L | CTA | 0.04 | 6 |
| L | CTC | 0.10 | 5 |
| L | CTG | 0.49 | 1 |
| L | CTT | 0.11 | 4 |
| L | TTA | 0.13 | 2 |
| L | TTG | 0.13 | 3 |
| M | ATG | 1.00 | 1 |
| N | AAC | 0.53 | 1 |
| N | AAT | 0.47 | 2 |
| P | CCA | 0.19 | 2 |
| P | CCC | 0.13 | 4 |
| P | CCG | 0.51 | 1 |
| P | CCT | 0.17 | 3 |
| Q | CAA | 0.33 | 2 |
| Q | CAG | 0.67 | 1 |
| R | AGA | 0.05 | 5 |
| R | AGG | 0.03 | 6 |
| R | CGA | 0.07 | 4 |
| R | CGC | 0.37 | 1 |
| R | CGG | 0.11 | 3 |
| R | CGT | 0.36 | 2 |
| S | AGC | 0.27 | 1 |
| S | AGT | 0.16 | 2 |
| S | TCA | 0.14 | 6 |
| S | TCC | 0.15 | 3 |
| S | TCG | 0.15 | 4 |
| S | TCT | 0.15 | 5 |
| T | ACA | 0.15 | 4 |
| T | ACC | 0.41 | 1 |
| T | ACG | 0.27 | 2 |
| T | ACT | 0.17 | 3 |
| V | GTA | 0.16 | 4 |
| V | GTC | 0.21 | 3 |
| V | GTG | 0.37 | 1 |
| V | GTT | 0.26 | 2 |
| W | TGG | 1.00 | 1 |
| Y | TAC | 0.43 | 2 |
| Y | TAT | 0.57 | 1 |
| Stop | TAA | 0.59 | 1 |
| Stop | TAG | 0.09 | 3 |
| Stop | TGA | 0.32 | 2 |

The “Frequency” column contains the percentage proportion of the respective codon relative to the respective amino acid, while the “Rank” column contains the rank of the respective codons. The “Rank” value defines the frequency of the respective codon within an amino acid. Where there are two or more identical frequency values within an amino acid, the ranks of the equally frequent codons are additionally allocated alphabetically. The “Rank” column thus contains the key.

In the example, the alphabetically sorted codons for alanine (GCA, GCC, GCG, GCT) have the order of precedence 3, 2, 1, 4 or 3214.

For amino acids with one codon (M,W), there is only one possibility for order of precedence (1).

For amino acids with two codons (C, D, E, F, H, K, N, Q, Y), there are two possibilities for order of precedence (12, 21).

For amino acids with three codons (I, stop), there are six possibilities for order of precedence (123, 132, 213, 231, 312, 321).

For amino acids with four codons (A, G, P, T, V), there are 24 possibilities for order of precedence (1234, 1243, 1324 . . . 4231, 4312, 4321).

For amino acids with six codons (L, R, S), there are 720 possibilities for order of precedence (123456, 123465, 123546, . . . 654231, 654312, 654321).

On the basis of these figures, it becomes clear that there are 1²×2⁹×6²×24⁵×720³=5.48×10¹⁹ different combinations of order of precedence. This is thus the number of possible keys.

For each amino acid group (one, two, three, four, six codons), an ascending list of all possible orders of precedence is drawn up and consecutively numbered in binary. This is shown by way of example for the 24 possible orders of precedence of the amino acids with four codons (A, G, P, T, V):

| Order of | | |
|---|---|---|
| precedence | Decimal | Binary |
| 1234 | 00 | 00000 |
| 1243 | 01 | 00001 |
| 1324 | 02 | 00010 |
| 1342 | 03 | 00011 |
| 1423 | 04 | 00100 |
| 1432 | 05 | 00101 |
| 2134 | 06 | 00110 |
| 2143 | 07 | 00111 |
| 2314 | 08 | 01000 |
| 2341 | 09 | 01001 |
| 2413 | 10 | 01010 |
| 2431 | 11 | 01011 |
| 3124 | 12 | 01100 |
| 3142 | 13 | 01101 |
| 3214 | 14 | 01110 |
| 3241 | 15 | 01111 |
| 3412 | 16 | 10000 |
| 3421 | 17 | 10001 |
| 4123 | 18 | 10010 |
| 4132 | 19 | 10011 |
| 4213 | 20 | 10100 |
| 4231 | 21 | 10101 |
| 4312 | 22 | 10110 |
| 4321 | 23 | 10111 |

0 binary digits are required for the binary coding of the order of precedence of amino acid with one codon.

1 binary digit (decimal 0=binary 0 & decimal 1=binary 1) is required for the binary coding of the order of precedence of amino acids with two codons.

3 binary digits (decimal 0=binary 000 & decimal 5=binary 101) are required for the binary coding of the order of precedence of amino acids with three codons.

5 binary digits (decimal 0=binary 00000 & decimal 23=binary 10111) are required for the binary coding of the order of precedence of amino acids with four codons.

10 binary digits (decimal 0=binary 0000000000 & decimal 719=binary 1011001111) are required for the binary coding of the order of precedence of amino-acids with six codons.

A specific binary number may accordingly be assigned to each order of precedence of the alphabetically sorted amino acids. The entirety of the binary numbers represents the specific codon usage table which is used for the steganographic method.

| | Order of | | Only 4 fold & 6 |
|---|---|---|---|
| Amino acid | precedence | Binary | fold |
| A | 3214 | 01110 | 01110 |
| C | 12 | 0 | |
| D | 21 | 1 | |
| E | 12 | 0 | |
| F | 21 | 1 | |
| G | 4132 | 10011 | 10011 |
| H | 21 | 1 | |
| I | 321 | 101 | |
| K | 12 | 0 | |
| L | 651423 | 1010111100 | 1010111100 |
| M | 1 | | |
| N | 12 | 0 | |
| P | 2413 | 01010 | 01010 |
| Q | 21 | 1 | |
| R | 564132 | 1001010011 | 1001010011 |
| S | 126345 | 0000010010 | 0000010010 |
| T | 4123 | 10010 | 10010 |
| V | 4312 | 10110 | 10110 |
| W | 1 | | |
| Y | 21 | 1 | |
| Stop | 132 | 001 | |

The entire 70-digit binary sequence of the codon usage table of this example accordingly reads:
0111001011001111010101011110000101011001010011000001001010010 101101001

In order to translate this binary sequence into a nucleotide sequence, each nucleobase is assigned a fixed, two-digit binary value: A=00, C=01, G=10, T=11

Using this key, the binary sequence can be translated into a 35-digit nucleotide sequence

CTAGTATTCCCCTGACCCGCCATAACAGGCCCGGC

If only amino acids with four or six codons are used during the steganographic embedding of information into the coding sequence, it is sufficient to restrict oneself to these amino acids when depositing the codon usage table. The relevant binary numbers are stated in the above table in the “Only 4 fold & 6 fold” column and together give rise to the 56-digit binary sequence:
01110100111010111100010101001010011000001001010010101100

Using the above-mentioned key, this may be translated into the following 28-digit nucleotide sequence:

CTCATGGTTACCCAGGCGAAGCCAGGTA

As already mentioned, the binary sequence may furthermore be encrypted with a password using conventional encryption algorithms prior to translation into a nucleotide sequence.

Translation of the nucleotide sequence back into a binary sequence and an order of precedence (key) proceeds in the reverse order in a similar manner to the described method.

### Example 3

### Study into the Expression of E. coli

Construct eGFP(opt):

The open reading frame for enhanced green fluorescent protein (eGFP) was optimised for expression in E. coli. In so doing, a codon adaptation index (CAI) of 0.93 and a GC content of 53% were achieved.

Construct eGFP(msg):

According to the invention, the message “AEQUOREA VICTORIA.” was embedded into the optimised DNA sequence, the key used being the codon usage table (CUT) of E. coli and the only codons used to accommodate the bits being those which have a degree of degeneracy of 4 or 6 and thus encode the amino acids A, G, P, T, V, L, R, S. Embedding the 18×6=10⁸ bit long message results in 71 nucleotide substitutions, so modifying the sequence by 10%. The CAI changes to 0.84, the GC content to 47%.

FIG. 6 shows an alignment of the two sequences eGFP(opt) and eGFP(msg).

Both genes were produced synthetically and, via NdeI/HindIII, ligated into the expression vector pEG-His. The proteins consequently contain a C terminal 6×His-tag.

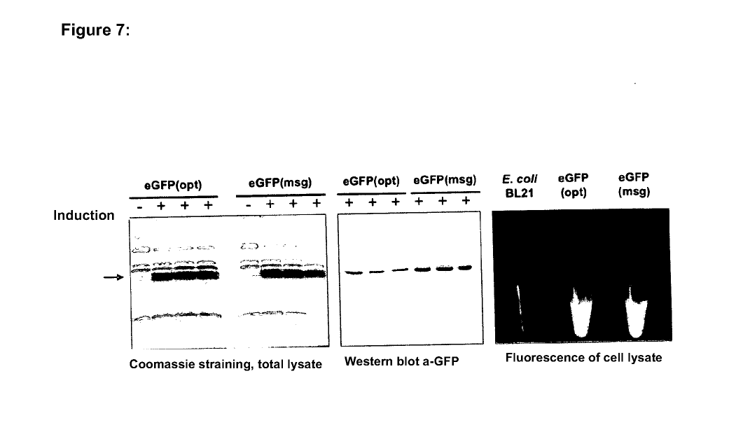

Both genes, eGFP(opt) and eGFP(msg) were expressed in E. coli and analysed by Coomassie gel, Western blot (with a GFP-specific antibody) and fluorescence. The results are shown in FIG. 7. It was found that eGFP(msg) exhibits expression which is better by a factor of approx. 2 than eGFP(opt). This increase in expression is a random effect and not the rule (according to studies with other genes). What is important to note is that expression does not suffer from the embedding of the message.

### Example 4

### Study of Expression in Human Cells

Construct EMG1 (opt):

The open reading frame for the human gene EMG1 nucleolar protein homologue was optimised for expression in human cells. In so doing, a codon adaptation index (CAI) of 0.97 and a GC content of 64% were achieved.

Construct EMG1(msg):

According to the invention, the message “GENEARTAG PAT U.S. Pat. No. 1,234,567” was embedded into the optimised DNA sequence, the key used being the codon usage table (CUT) of H. sapiens and the only codons used to accommodate the bits being those which have a degree of degeneracy of 4 or 6 and thus encode the amino acids A, G, P, T, V, L, R, S. Embedding the 24×6=144 bit long message results in 92 nucleotide substitutions, so modifying the sequence by 12%. The CAI changes to 0.87, the GC content to 59%.

Construct EMG1(enc):

The message “GENEARTAG PAT U.S. Pat. No. 1,234,567” was firstly encrypted using the conventional polyalphabetic Vigenère method (after Blaise de Vigenere, 1586) with the password “Secret”, so generating the character string “:JQWF&G % DY %$4Y#′XE %87G;K” from the message. In addition to the very simple and insecure Vigenere method, in which a plaintext letter is replaced by different ciphertext letters depending on its position in the text, it is in principle possible to use any other encryption method. According to the invention, the encrypted character string “:JQWF&G % DY %$4Y#′XE %87G;K” was embedded into the optimised DNA sequence, the key used being the codon usage table (CUT) of H. sapiens and the only codons used to accommodate the bits being those which have a degree of degeneracy of 4 or 6 and thus encode the amino acids A, G, P, T, V, L, R, S. Embedding the 24×6=144 bit long message results in 93 nucleotide substitutions, so modifying the sequence by 12%. Here too, the CAI changes to 0.87, the GC content to 59%.

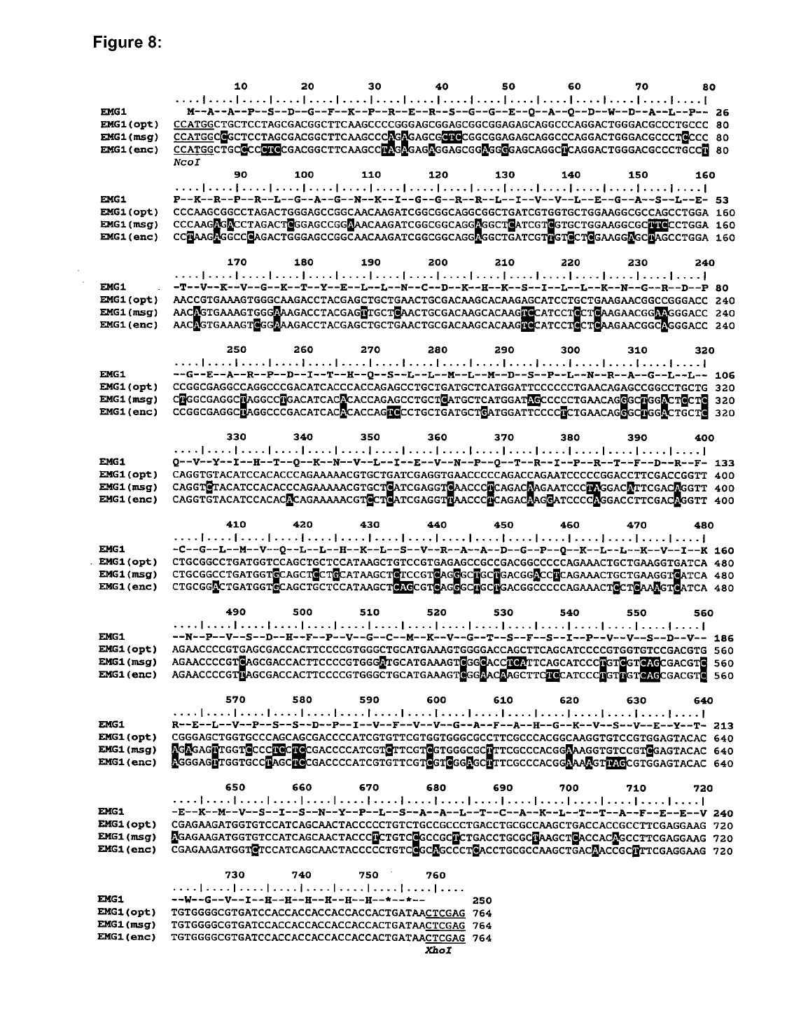

FIG. 8 shows an alignment of the sequences of EMG1(opt), EMG1(msg) and EMG1 (enc).

All three genes were produced synthetically and, via NcoI/XhoI, ligated into the vector pTriEx1.1 which permits expression in mammalian cells.

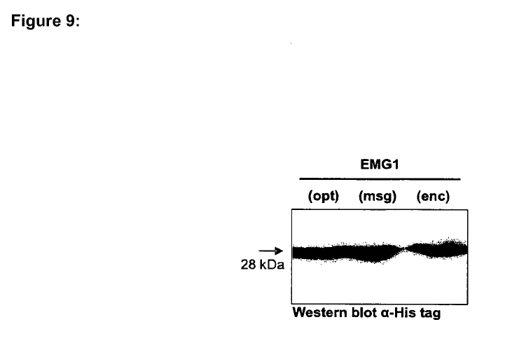

Human HEK-293T cells were transfected with the three constructs EMG1(opt), EMG1(msg) and EMG1(enc) and harvested after 36 h. Expression of EMG1 was detected by Western blot analysis (with a His-specific antibody). All three construct. exhibit a comparable strength of expression. The results are shown in FIG. 9.

1. A method for designing nucleic acid sequences in which items of information are contained, which comprises the steps:
(a) assigning a first specific value to at least one first nucleic acid codon from a group of degenerate nucleic acid codons which encode the same amino acid,
assigning a second specific value to at least one second nucleic acid codon from the group,
optionally assigning one or more further values to in each case at least one further nucleic acid codon from the group,
in which the first and second and optionally further values are in each case allocated at least once within the group of codons which encode the same amino acid;
(b) providing an item of information to be stored as a series of n values which are in each case selected from first and second and optionally further values;
(c) providing a starting nucleic acid sequence, wherein the sequence comprises n degenerate codons to which first and second and optionally further values are assigned according to (a), in which n is an integer ≧1; and
(d) designing a modified sequence of the nucleic acid sequence from (c), in which, at the positions of the n degenerate codons of the starting nucleic acid sequence, in each case one nucleic acid codon is selected from the group of degenerate codons which encode the same amino acid, to which codon there corresponds a value due to the assignment from (a) so that the series of the values assigned to the n codons results in the item of information to be stored.

2. The method according to claim 1, in which the amino acids in step (a) are selected from six-fold encoded amino acids, such as leucine, serine, arginine, and/or four-fold encoded amino acids, such as alanine, glycine, valine, proline.

3. The method according to claim 1, in which, in step (a), first, second or optionally further values are assigned to all the codons which encode the same amino acid or stop.

4. The method according to claim 1, in which first and second values but no further values are assigned in step (a), and the item of information in step (b) is provided in binary form.

5. The method according to claim 4, in which the first and second values are in each case allocated multiple times, in particular an equal amount of times, within the group of degenerate nucleic acid codons which encode the same amino acid or stop.

6. The method according to claim 1, in which the assignment of a first or second or optionally further value to a nucleic acid codon within the group of degenerate codons which encode the same amino acid or stop takes place in step (a) in a manner dependent on the frequency of use of the codon in a specific organism.

7. The method according to claim 1, in which the starting nucleic acid is a coding DNA strand.

8. The method according to claim 1, in which the starting nucleic acid encodes a polypeptide and the modified sequence designed in step (d) encodes the same polypeptide.

9. The method according to claim 1, in which the item of information to be stored comprises graphic, text or image data.

10. The method according to claim 1, in which text data in step (b) are represented in binary form by means of the ASCII code.

11. The method according to claim 1, in which the start and/or end of the item of information to be stored are marked in the polynucleotide derivative.

12. The method according to claim 1, further comprising the step
(e) producing the modified sequence designed in step (d).

13. The method according to claim 12, in which the modified sequence is produced in step (e) by mutation from the starting sequence, in particular by substitution.

14. The method according to claim 12, in which the modified sequence is produced synthetically in step (e).

15. The method according to claim 1, in which the item of information to be stored is encrypted before it is converted into a series of n values.

16. The method according to claim 1, in which a key for the assignment according to step (a) is itself encrypted and stored in a nucleic acid.

17. The method according to claim 16, in which the key is stored in the nucleic acid derivative from step (d) or in another nucleic acid.

18. A modified nucleic acid sequence, obtainable by a method according to claim 1.

19. A modified nucleic acid, obtainable by a method according to claim 14.

20. A vector, comprising a modified nucleic acid according to claim 19.

21-26. (canceled)
